(19) United States
(12) Patent Application Publication
(10) Pub. No.: US 2018/0156707 A1
(43) Pub. Date: Jun. 7, 2018

(54) PORTABLE HYDROSTATIC TESTER

(71) Applicant: Atacama, Inc., Alameda, CA (US)

(72) Inventor: Siyuan Xing, Davis, CA (US)

(73) Assignee: Atacama, Inc., Alameda, CA (US)

(21) Appl. No.: 15/832,514

(22) Filed: Dec. 5, 2017

Related U.S. Application Data
(60) Provisional application No. 62/431,308, filed on Dec. 7, 2016.

(51) Int. Cl. G01N 15/08; G01N 33/36

(52) U.S. Cl. CPC G01N 15/082; G01N 33/367

(57) ABSTRACT

The present disclosure provides a portable hydrostatic tester for hydrostatic pressure resistance of a fabric without the need for a power source and the need for cutting the fabric before testing. In some embodiments, the hydrostatic tester comprises (1) a supporting structure comprising a test opening; (2) a connection tube connected to the test opening; (3) a liquid column connected to the connection tube; and (4) a reservoir configured to contain a liquid that is connected to one or both of the liquid column and the connection tube. In some embodiments, the reservoir is configured such that the liquid is forced by gravity through the test opening via the connection tube at a hydrostatic pressure dependent upon the elevation of the reservoir. The present disclosure also provides a method of testing the hydrostatic pressure resistance of a fabric using the hydrostatic tester described above.

### CROSS REFERENCE TO RELATED APPLICATIONS

This application claims the priority benefit of U.S. Provisional Application Ser. No. 62/431,308, filed Dec. 7, 2016, which is hereby incorporated by reference in its entirety.

### STATEMENT OF GOVERNMENT SUPPORT

This invention was made with Government support under Small Business Innovation Research (SBIR) Grant No. 1556133 awarded by the National Science Foundation. The Government has certain rights in this invention.

### FIELD

The present disclosure relates to portable hydrostatic testers (e.g., for hydrostatic testing of fabrics or other materials), as well as methods of use related thereto.

### BACKGROUND

The ability of a material to resist hydrostatic pressure is critical in fabric development, especially in waterproof fabric development. Generally, a fabric with a high hydrostatic pressure provides a robust waterproof capacity in practical applications. Therefore, an apparatus to test the hydrostatic pressure resistance of a fabric and methods of use related thereto is important in fabric development. Existing hydrostatic testing devices are mostly designed for laboratory settings, and not easily portable due to their sizes and shapes. Additionally, existing devices often rely on power-driven devices, such as electronically-driven motors and pumps, to apply the hydrostatic pressure onto the tested material. This reliance requires a significant and continuous source of power, such as an electrical outlet. Moreover, existing devices are commonly designed in a way that forces the user to pre-cut the fabric to a sample size that fits a specific area on the device. This limits the size of the sample that can be tested. For example, large samples, such as full-scale products including but not limited to a completed shirt or a full jacket, cannot be easily tested without the pre-cut process. Similarly, a small and specific hydrophobic region on a fabric, such as a 2 mm diameter circular area, cannot be easily tested either. Other testing devices, such as the apparatus for whole garment leak testing described in U.S. Pat. No. 5,390,531 are able to test completed garments, but not specific areas of the garment.

Therefore, a need exists for a portable hydrostatic tester that is able to test a specific portion of a fabric without the need to pre-cut or damage the fabric, and is able to provide hydrostatic pressure without the need for a power source.

### BRIEF SUMMARY

To meet these and other demands, the present disclosure provides a gravity-driven hydrostatic tester for fabrics and other materials, and methods of using thereof. The tester comprises a supporting structure within which a portion of a large testing sample may be placed to test a specific portion of the testing sample without destroying or altering the sample. Further, the tester comprises a quantifiable measurement of the ability of the sample to prevent the penetration of a pressurized liquid, such as a liquid column. Additionally, the tester comprises a reservoir connected to the supporting structure via a connection tube. Upon elevation of the reservoir, liquid fills the connection tube and begins to fill the liquid column. The liquid accumulated in the liquid column is forced by gravity to apply a hydrostatic pressure on the sample via the connection tube and a test opening in a supporting structure. The hydrostatic pressure resistance of the sample is indicated by penetration of the liquid through the sample, and can be measured by the liquid level in the liquid column. This configuration incorporates portability, versatility, and affordability, and is particularly useful for quality control testing of a fabric during manufacturing. This configuration also saves time and improves the testing efficiency.

In certain aspects, the present disclosure provides a device for hydrostatic testing of a fabric, comprising: (1) a supporting structure comprising a test opening, wherein the supporting structure is configured to support the fabric; (2) a connection tube comprising a first end and a second end, wherein the first end is connected to the test opening; (3) a liquid column connected to the second end of the connection tube; and (4) a reservoir configured to contain a liquid, wherein the reservoir is connected to one or both of the liquid column and the connection tube, and wherein the reservoir is configured such that the liquid is forced by gravity through the test opening via the connection tube at a hydrostatic pressure dependent upon the volume of water contained in the connection tube and liquid column. In some embodiments, the reservoir is connected to one or both of the liquid column and the connection tube via an inlet tube. In some embodiments, a junction between the inlet tube and the liquid column and/or the connection tube is positioned lower than the test opening. In some embodiments, the inlet tube is positioned lower than the test opening. In some embodiments, the inlet tube further comprises a valve configured to adjust the flow of the liquid from the reservoir. In some embodiments, the inlet tube comprises polyurethane, vinyl, or plastic. In some embodiments, the liquid column is configured such that the level of the liquid within the liquid column indicates the hydrostatic pressure. In some embodiments, at least a portion of the liquid column is substantially transparent. In some embodiments, the liquid column further comprises an indication of hydrostatic pressure. In some embodiments, the liquid column is a cylinder. In some embodiments, the liquid column is a rectangular prism. In some embodiments, the liquid column has a first end and a second end, wherein the first end is open, and wherein the second end is connected to the second end of the connection tube. In some embodiments, the device further comprises a top plate, wherein the top plate is configured to be positioned atop one or both of the fabric and the supporting structure. In some embodiments, the top plate comprises a window for viewing the fabric. In some embodiments, at least a portion of the window is configured to be aligned with the test opening via an alignment structure. In some embodiments, the alignment structure comprises a guide-pin with an alignment hold, a stop-block, or a magnet. In some embodiments, the supporting structure and the top plate are compressed via a compression mechanism. In some embodiments, the compression mechanism comprises a c-clamp, a nut and a bolt, an elastic band, or a magnet. In some embodiments, the top plate comprises wood, acrylic, polycarbonate, metal, or glass. In some embodiments, the supporting structure comprises wood, acrylic, polycarbonate, metal, or glass. In some embodiments, the test opening further comprises a sealing structure configured to form a seal between the fabric and the test opening. In some embodiments, the sealing structure comprises a gasket. In some embodiments, the sealing structure comprises a compressible material. In some embodiments, the compressible material comprises silicone, rubber, or foam. In some embodiments, the test opening has a diameter of between about 1 mm and about 10 cm. In some embodiments, the test opening has a diameter between about 2 mm and about 3 mm. In some embodiments, the test opening comprises wood, acrylic, polycarbonate, metal, or glass. In some embodiments, the reservoir is a cylinder. In some embodiments, the reservoir further comprises an indication of the liquid level inside the reservoir. In some embodiments, the reservoir comprises glass, PVC, metal, PET, or polycarbonate. In some embodiments, the connection tube comprises plastic or metal. In some embodiments, the metal is aluminum, aluminum alloy, copper, brass, or steel (e.g., stainless steel). In some embodiments, the liquid is water. In some embodiments, the device lacks a pump or motor. In some embodiments, the liquid column contains a liquid.

In another aspect, the present disclosure provides a method for testing the hydrostatic pressure resistance of a fabric, comprising a) providing a device, the device comprising: (i) a supporting structure comprising a test opening, wherein the supporting structure is configured to support the fabric; (ii) a connection tube comprising a first end and a second end, wherein the first end is connected to the test opening; (iii) a liquid column connected to the second end of the connection tube; and (iv) a reservoir containing a liquid; b) placing the fabric on the test opening; and c) elevating the reservoir such that the liquid fills the connection tube and at least a portion of the liquid column, wherein the liquid is forced by gravity through the test opening via the connection tube at a hydrostatic pressure dependent upon the volume of liquid in the connection tube and the liquid column. In some embodiments, the method further comprises recording the liquid level within one or both of the liquid column and the reservoir after step c). In some embodiments, the method further comprises forming a seal between the fabric and the test opening between steps b) and c). In some embodiments, the seal is formed via a sealing structure. In some embodiments, the sealing structure comprises a gasket. In some embodiments, the sealing structure comprises a compressible material. In some embodiments, the compressible material comprises silicone, rubber, or foam. In some embodiments, the device is positioned on a substantially horizontal surface. In some embodiments, the liquid column is in a substantially vertical position. In some embodiments, the liquid is water. In some embodiments, the reservoir is connected to one or both of the liquid column and the connection tube via an inlet tube. In some embodiments, a junction between the inlet tube and the liquid column and/or the connection tube is positioned lower than the test opening. In some embodiments, the inlet tube is positioned lower than the test opening. In some embodiments, the inlet tube further comprises a valve configured to adjust the flow of the liquid from the reservoir. In some embodiments, the inlet tube comprises polyurethane, vinyl, or plastic. In some embodiments, the liquid column is configured such that the level of the liquid within the liquid column indicates the hydrostatic pressure. In some embodiments, at least a portion of the liquid column is substantially transparent. In some embodiments, the liquid column further comprises an indication of hydrostatic pressure. In some embodiments, the liquid column is a cylinder. In some embodiments, the liquid column is a rectangular prism. In some embodiments, the liquid column has a first end and a second end, wherein the first end is open, and wherein the second end is connected to the second end of the connection tube. In some embodiments, the device further comprises a top plate, wherein the top plate is configured to be positioned atop one or both of the fabric and the supporting structure. In some embodiments, the top plate comprises a window for viewing the fabric. In some embodiments, at least a portion of the window is configured to be aligned with the test opening via an alignment structure. In some embodiments, the alignment structure comprises a guide-pin with an alignment hole, a stop-block, or a magnet. In some embodiments, the supporting structure and the top plate are compressed via a compression mechanism. In some embodiments, the compression mechanism comprises a c-clamp, a nut and a bolt, an elastic band, or a magnet. In some embodiments, the top plate comprises wood, acrylic, polycarbonate, metal, or glass. In some embodiments, the supporting structure comprises wood, acrylic, polycarbonate, metal, or glass. In some embodiments, the test opening has a diameter of between about 1 mm and about 10 cm. In some embodiments, the test opening has a diameter between about 2 mm and about 3 mm. In some embodiments, an edge of the test opening comprises wood, acrylic, polycarbonate, metal, or glass. In some embodiments, the reservoir is a cylinder. In some embodiments, the reservoir further comprises an indication of the liquid level inside the reservoir. In some embodiments, the reservoir comprises glass, PVC, metal, PET, or polycarbonate. In some embodiments, the connection tube comprises plastic or metal. In some embodiments, the metal is aluminum, aluminum alloy, copper, brass, or steel (e.g., stainless steel). In some embodiments, the device lacks a pump or motor. In some embodiments, the liquid column contains a liquid.

It is to be understood that one, some, or all of the properties of the various embodiments described above and herein may be combined to form other embodiments of the present invention. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A & 1B show side (FIG. 1A) and top (FIG. 1B) views of a portable hydrostatic tester in accordance with some embodiments.

FIG. 2A shows a top view of a supporting structure without a top plate, revealing a view of the test opening, in accordance with some embodiments.

FIG. 2B shows a top view of a supporting structure with a top plate in place, in accordance with some embodiments.

FIGS. 3A & 3B show a portable hydrostatic tester with a fabric attached for testing in accordance with some embodiments.

FIG. 4 shows a portable hydrostatic tester with a top plate that includes optional alignment structures in accordance with some embodiments.

FIG. 5 shows a portable hydrostatic tester with a top plate that includes optional alignment structures in accordance with some embodiments.

### DETAILED DESCRIPTION

### I. Portable Hydrostatic Testers

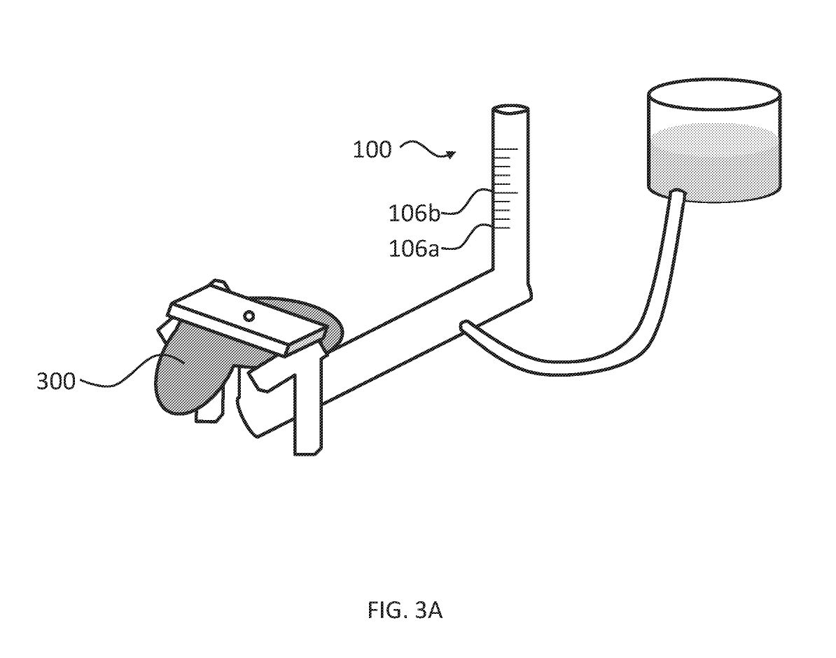

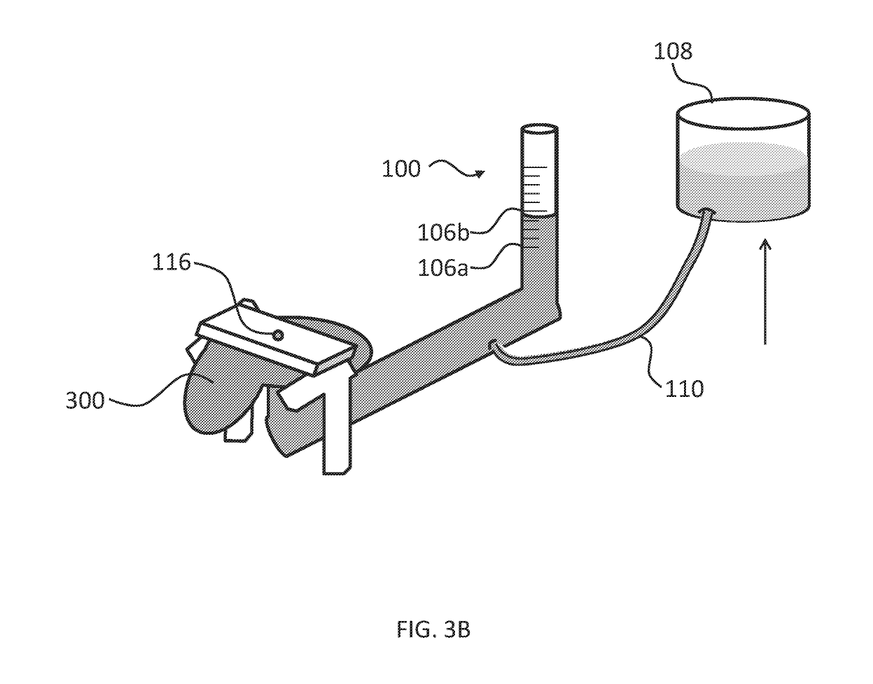

Certain aspects of the present disclosure relate to portable hydrostatic testers. Such testers may find use, e.g., in testing the ability of a fabric to resist hydrostatic pressure. In some embodiments, a gravity-driven hydrostatic testing device of the present disclosure comprises (1) a supporting structure comprising a test opening, wherein the supporting structure is configured to support the fabric; (2) a connection tube comprising a first end and a second end, wherein the first end is connected to the test opening; (3) a liquid column connected to the second end of the connection tube; and (4) a reservoir configured to contain a liquid, wherein the reservoir is connected to one or both of the liquid column and the connection tube. The hydrostatic testing device is configured to allow the liquid to be forced by gravity through the test opening via the connection tube at a hydrostatic pressure dependent upon the volume of water contained in the connection tube and liquid column (e.g., the level of liquid in the liquid column; see FIG. 3B). The hydrostatic pressure sustained by the sample can be indicated through a quantifiable measurement, such as the liquid column. This hydrostatic tester improves upon existing apparatuses and methods of testing a sample by eliminating the need for a power source to apply pressure, increasing portability, enabling the testing of specific portions of full-scale garments and large samples without the need to pre-cut or damage the sample, and providing the capacity to measure the hydrostatic pressure of a small area that would be difficult to cut from a larger sample.

As used herein and commonly known in the art, a hydrostatic pressure resistance of a material may refer to a measurement metric used to characterize the maximum constant hydrostatic pressure a porous material can withstand before it leaks. In existing materials, the hydrostatic pressure resistance equals the maximum impact pressure the fabric can withstand.

Although waterproof fabric is used as an example herein to illustrate the device and methods of using the device, it should be noted that the device can be used to test a broad range of porous materials. These can include, without limitation, hydrophobic fabrics, fabrics treated with a hydrophobic material on at least one side, patterned fabrics, non-woven materials, paper, sponges, and polymer membranes.

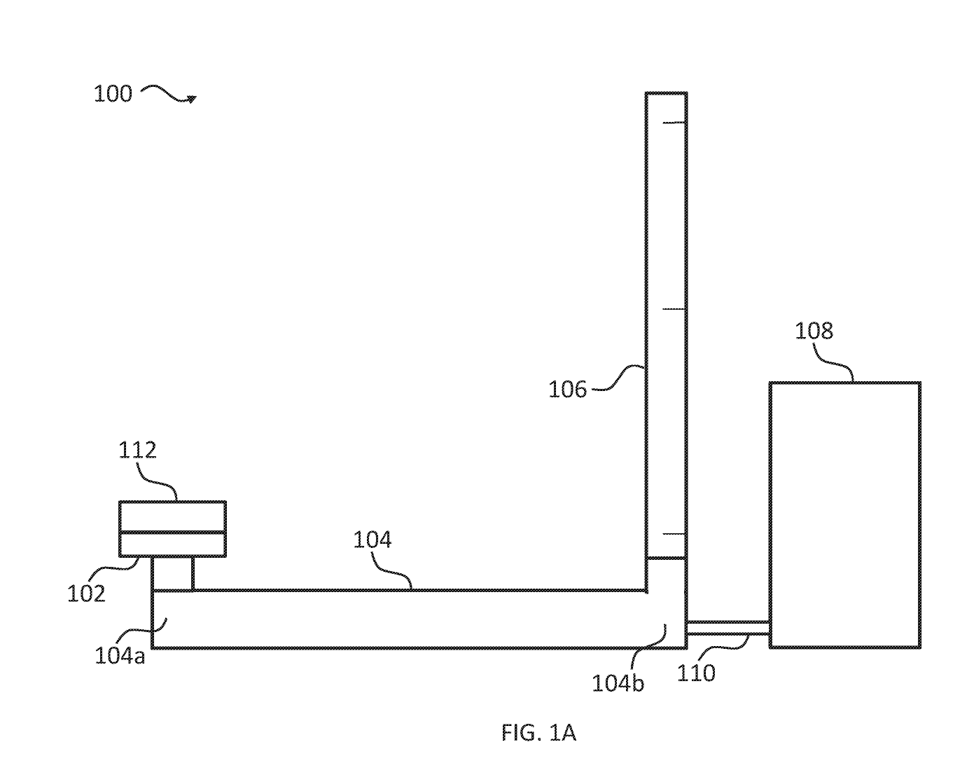

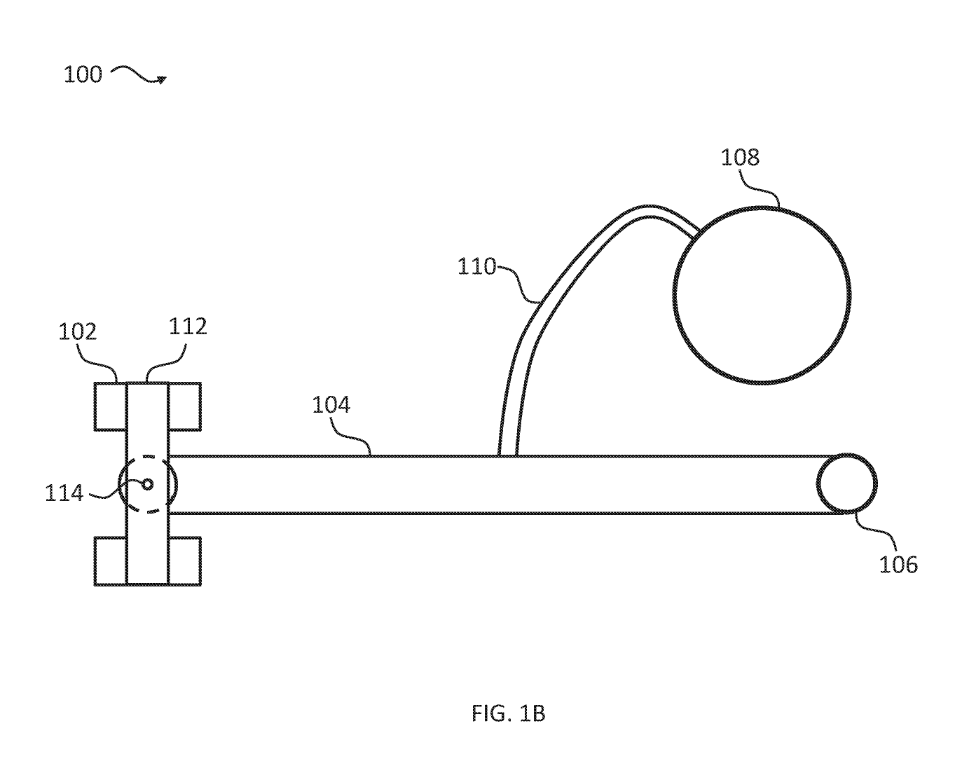

Exemplary portable hydrostatic tester 100 and optional aspects thereof are illustrated in FIGS. 1A-5. A side view of tester 100 is shown in FIG. 1A. Tester 100 includes supporting structure 102. Supporting structure 102 includes a test opening (shown as 114 in FIG. 1B) and is configured to support a fabric or portion thereof for hydrostatic testing, e.g., as described herein and illustrated in FIGS. 3A & 3B. Connected to structure 102 is connection tube 104. One end of connection tube 104 is connected to the test opening, as shown by end 104a. Another end of connection tube 104 is connected to liquid column 106, as shown by end 104b. In some embodiments, a liquid column of the present disclosure is configured such that it is able to contain a liquid. In some embodiments, e.g., during hydrostatic testing, at least a portion of the liquid column contains a liquid. In some embodiments, e.g., when tester 100 is not filled with a liquid, and/or the reservoir is not elevated, the liquid column does not contain a liquid. In this example, inlet tube 110 connects connection tube 104 with reservoir 108. Reservoir 108 is configured to contain a liquid, e.g., water for hydrostatic testing. Optional top plate 112 is configured to be positioned atop the fabric and supporting structure 102, e.g., such that the fabric is placed between structure 102 and top plate 112 over test opening 114.

A top view of the portable hydrostatic tester is shown in FIG. 1B. Supporting structure 102 includes test opening 114. Top plate 112 is positioned atop the fabric and supporting structure 102. Top plate 112 optionally has a window that allows at least a portion of the fabric and/or a portion of the test opening to be viewed. This allows the operator to determine when the fabric has exceeded its hydrostatic pressure limit by observing when the liquid penetrates through the fabric. Supporting structure 102 is connected to one end of connection tube 104 (shown as 104a in FIG. 1A). Liquid column 106 is connected to another end of connection tube 104 (shown as 104b in FIG. 1A). In this example, inlet tube 110 connects connection tube 104 with reservoir 108. Reservoir 108 is configured to contain a liquid, e.g., water for hydrostatic testing.

Upon elevation of reservoir 108, the liquid within the reservoir is forced by gravity to flow from the reservoir through the test opening (shown as 114 in FIG. 1B) via inlet tube 110 and connection tube 104. The liquid, which is forced by gravity through test opening 114, contacts the fabric placed on supporting structure 102 with a specific hydrostatic pressure. The hydrostatic pressure applied to the fabric is dependent upon the volume of liquid in connection tube 104 and column 106, and can be indicated by the liquid level and/or volume within liquid column 106, which is proportional to the hydrostatic pressure of the liquid exiting the tester when leakage occurs. Optional top plate 112 positioned atop the fabric and/or supporting structure 102 provides additional support for the fabric during testing. When the liquid penetrates the fabric through the test opening, the liquid level and/or volume within liquid column 106 indicates the hydrostatic pressure resistance of the fabric, which equals the maximum impact pressure the fabric can withstand.

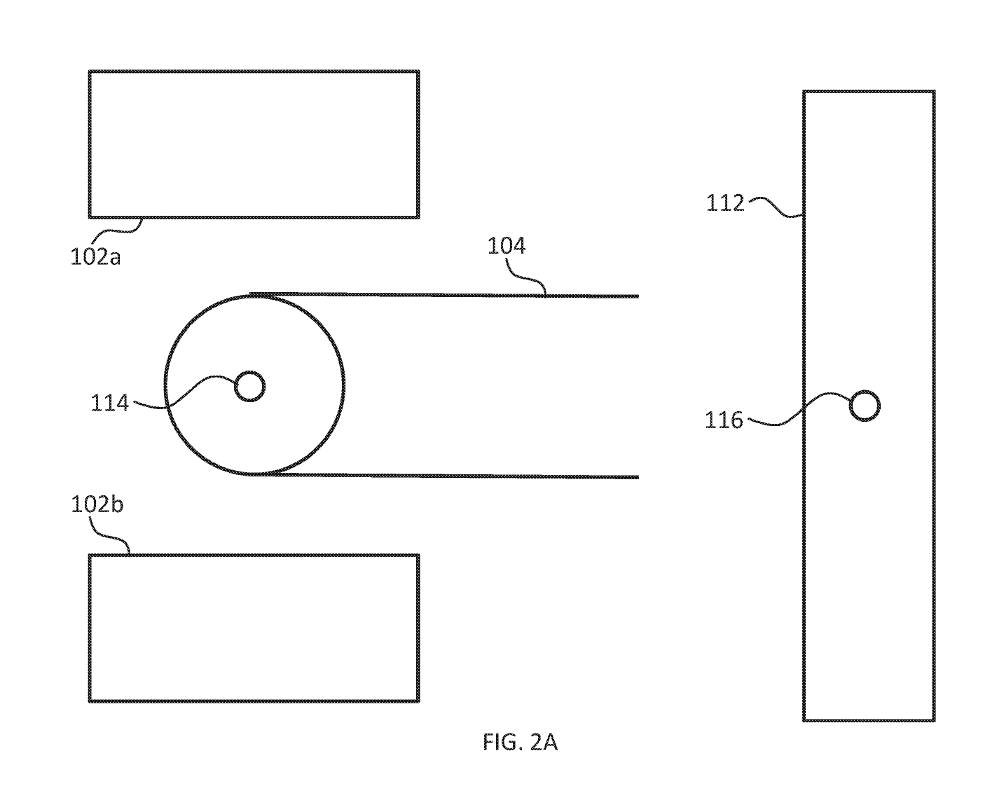

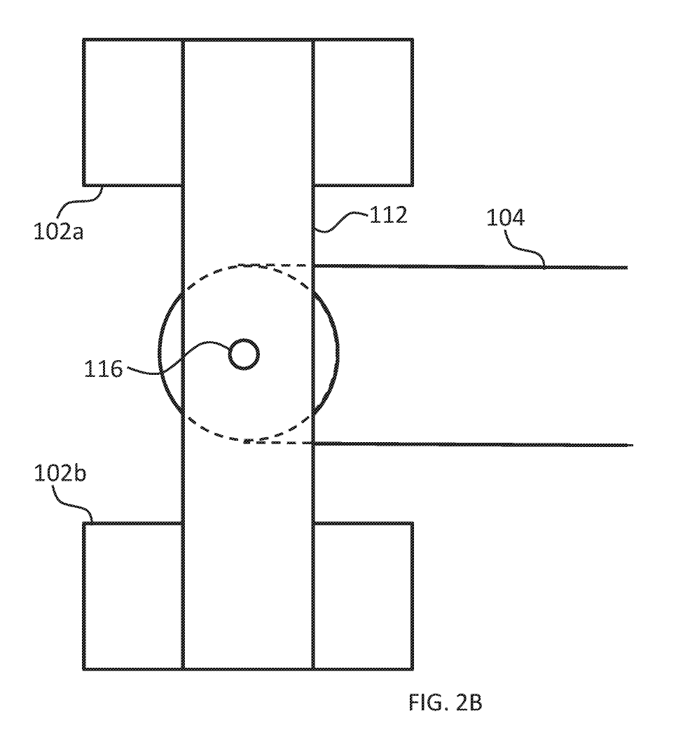

A top view of a supporting structure without a top plate is shown in FIG. 2A. Supporting structure includes test opening 114 that is connected with connection tube 104. In this example, supporting structure 102 includes two supporting ends, 102a and 102b. Ends 102a and 102b may be connected to tester 100, or they may be separate from the tester but positionable on either side of test opening 114. The fabric to be tested is placed atop test opening 114 and, optionally, one or both of 102a and 102b.

A top view of a supporting structure with a top plate in place is shown in FIG. 2B. Top plate 112 is configured to be positioned atop the fabric, test opening 114, supporting end 102a, and/or supporting end 102b, while at least a portion of window 116 is aligned with test opening 114. Window 116 of top plate 112 allows at least a portion of the fabric positioned atop test opening 114 to be viewed. When then liquid passes through the fabric via test opening 114 into window 116, the liquid level and/or volume of the liquid column indicates the hydrostatic pressure at which the fabric failed.

In some embodiments, the operator observes the hydrostatic pressure at which the liquid penetrates the fabric through a viewing window of the top plate. For example, the viewing window can be a hole in the top plate, and liquid in the hole indicates liquid penetration of the fabric. In other embodiments, the viewing window can be made of a clear material, and observable liquid contacting the top plate indicates liquid penetration of the fabric.

Portable hydrostatic tester 100 is shown in FIG. 3A with fabric 300 attached for testing. Fabric 300 is placed on the test opening (e.g., 114) of the supporting structure. As described above, top plate 112 is added on top of the fabric to provide extra support. In some embodiments, window 116 is aligned with the test opening, allowing at least a portion of the fabric to be observed. The fabric is larger than the supporting structure and is not pre-cut before testing. In this example, liquid column 106 has pre-calibrated markings (e.g., 106a and 106b) that provide quantitative measurements of the hydrostatic pressure dependent upon elevation of the reservoir. The operator records the liquid level in column 106 (e.g., using the pre-calibrated markings) when liquid penetration of the fabric is observed. After testing the hydrostatic pressure of one area on the fabric, the position of fabric can be changed to allow testing of a different area without cutting or damaging the fabric.

As shown in FIG. 3B, upon elevation of reservoir 108, liquid travels through inlet tube 110 and connection tube 104, exiting test opening 114. In FIG. 3B, the hydrostatic pressure of the liquid has exceeded the resistance of fabric 300. Thus, liquid penetrating through fabric 300 is visible in viewing window 116. If the viewing window comprises a hole, liquid can be observed leaking out of viewing window 116 after the hydrostatic pressure of the liquid has exceeded the resistance of fabric 300. This hydrostatic pressure is indicated by the level of liquid visible in liquid column 105, e.g., the pressure indicated by marking 106b.

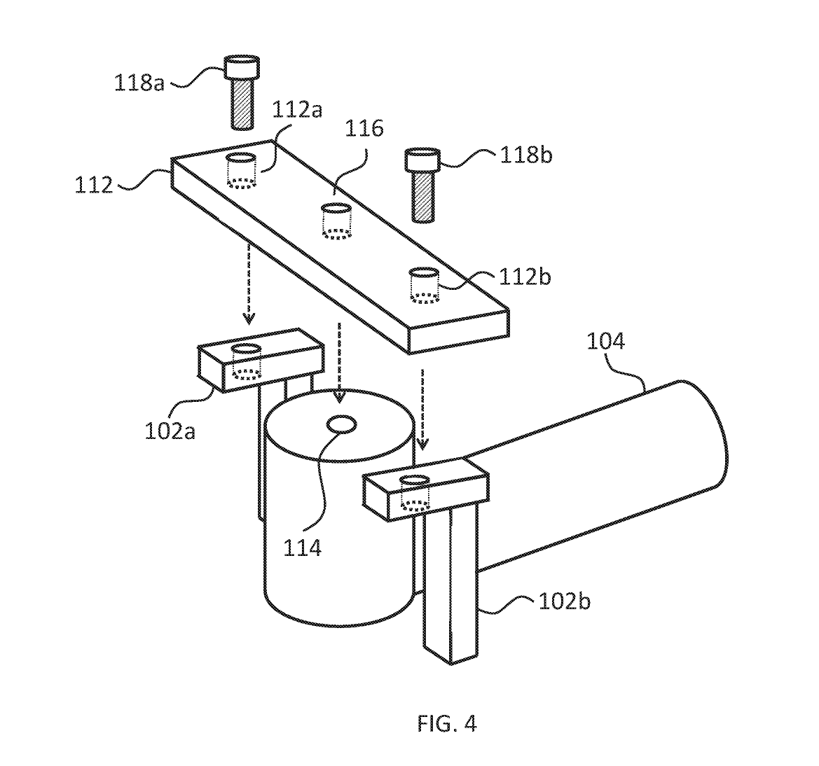

A portable hydrostatic tester with a top plate that includes optional alignment structures is shown in FIG. 4. In this example, supporting structure 102 has two supporting ends 102a and 102b. Supporting ends 102a and 102b each has a substantially vertical hole. Test opening 114 is located below top plate 112, and configured to support the fabric to be tested. In this example, top plate 112 comprises two alignment holes: 112a and 112b. These alignment holes are configured to be aligned with the substantially vertical holes in 102a and 102b, respectively. Guide pins 118a and 118b are configured to align and secure top plate 112 atop supporting structure 102 through alignment holes and the substantially vertical holes. Top plate 112 includes a window 116, and window 116 is configured to align with test opening 114 after top plate 112 is secured atop supporting structure 102 with guide pins. Upon elevation of the reservoir, the liquid applies a hydrostatic pressure to the fabric placed on supporting structure 102 via testing opening 114. The penetration of the liquid through the fabric can be observed through window 116, and the liquid level and/or volume within the liquid column can be recorded. Other alignment structures are described infra.

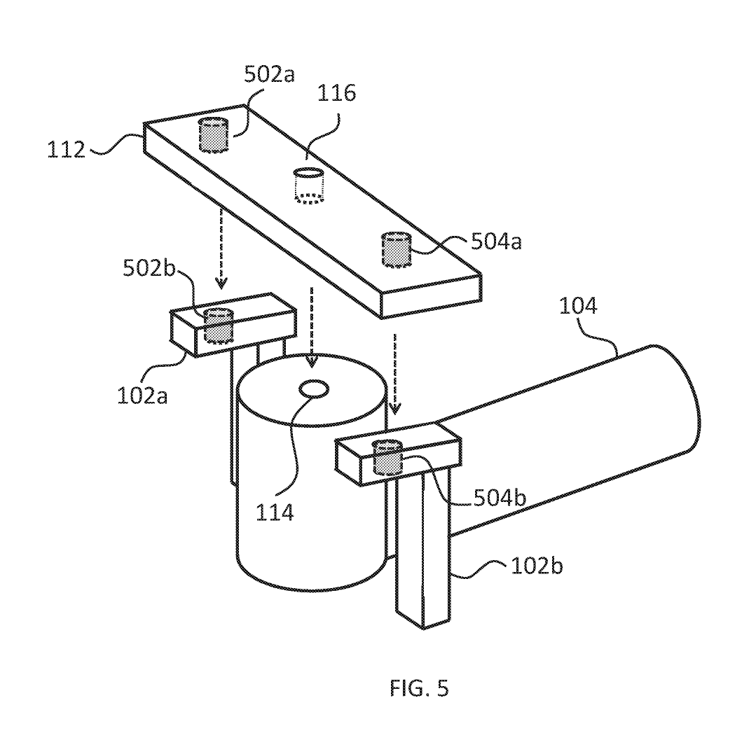

A portable hydrostatic tester with a top plate that includes another type of optional alignment structure is shown in FIG. 5. In this example, the device is the same as that illustrated in FIG. 4, but the alignment structure comprises magnets instead of alignment holes and guide pins. Shown in FIG. 5 are two pairs of magnets: 502a and 502b; and 504a and 504b. Magnet pairs 502a/502b and 504a/504b are configured to align and secure top plate 112 atop supporting structure 102 through attraction between the magnet pairs. Window 116 is configured to align with test opening 114 after top plate 112 is secured atop supporting structure 102 with magnet pairs 502a/502b and 504a/504b. Although two pairs of magnets are illustrated, other numbers of magnet pairs (e.g., one, three, four, five, six, seven, eight, nine, ten, or more) are possible.

In some embodiments, the reservoir is connected to the connection tube via an inlet tube. Exemplary embodiments are shown in FIG. 1A and FIG. 1B. Inlet tube 110 connects reservoir 108 to connection tube 104. In some embodiments, the inlet tube is joined at many points on the connection tube. In some embodiments, the reservoir is connected to the liquid column via an inlet tube. In some embodiments, the inlet tube is joined at many points on the liquid column. In some embodiments, the reservoir is connected to both the connection tube and the liquid column via one or more inlet tubes.

In some embodiments, the inlet tube is positioned lower than the test opening. Advantageously, this helps to prevent leakage when a sample is not being tested. In some embodiments, the inlet tube is connected to a portion of the reservoir that is lower than the geometric center of the reservoir to better utilize the capacity of the reservoir. An exemplary embodiment is shown in FIG. 1A. Inlet tube 110 is connected to reservoir 108 at a portion lower than the reservoir's geometric center.

In some embodiments, the inlet tube further comprises a valve configured to adjust the flow of the liquid from the reservoir. The valve may be placed within the inlet tube to adjust the liquid flow from the reservoir to the connection tube. In some embodiments, the valve can be used to turn on and turn off the liquid flow from the reservoir to the connection tube.

In some embodiments, the liquid column is configured such that the level of the liquid within the liquid column indicates the hydrostatic pressure. The hydrostatic pressure can be determined by measuring the liquid level or/and liquid volume within the liquid column. In some embodiments, at least a portion of the liquid column is substantially transparent or semi-transparent, e.g., such that the liquid level in the column can be observed. This portion of the liquid column can be in the form of small portal views, and can be continuous for greater accuracy. The liquid level can also be measured by pre-calibrated markings on the liquid column. In some embodiments, the liquid column further comprises an indication of hydrostatic pressure. Pre-calibrated markings on the liquid column can indicate the liquid volume inside the liquid column, which indicates the hydrostatic pressure. For example, a large portion of the liquid column can be substantially transparent or semi-transparent and can include several pre-calibrated markings for determining hydrostatic pressure. Alternatively, the tester can be configured to indicate a smaller number of potential pressures (e.g., a single hydrostatic pressure of interest) with a single marking and/or substantially transparent or semi-transparent portion. External measurement methods, such as a ruler, can also indicate the hydrostatic pressure.

The liquid column can be in any shape that can contain a liquid. In some embodiments, the liquid column is a cylinder. An exemplary embodiment is shown in FIGS. 3A & 3B. In other embodiments, the liquid column is a rectangular prism.

In some embodiments, the liquid column has a first end and a second end, wherein the first end is open, and wherein the second end is connected to the second end of the connection tube. An exemplary embodiment is shown in FIG. 1A. Liquid column 106 has two ends, and the second end is connected to the second end of connection tube 104b. The first end is open to allow the device to be connected to the atmosphere, and allow more accurate measurement of the hydrostatic pressure applied to the fabric.

In some embodiments, the device comprises a top plate, wherein the top plate is configured to be positioned atop one or both of the fabric and the supporting structure. The top plate provides extra support for the fabric during testing. In one aspect, the top plate helps immobilize the fabric by compressing the fabric with its own weight. The weight of the top plate allows the fabric to remain substantially stationary when a hydrostatic pressure is applied. The compression between the top plate and the fabric facilitates accurate measurements of the hydrostatic pressure.

In some embodiments, the top plate comprises a window for viewing the fabric. The window allows observation of the fabric during testing, and can take many forms. For example, the window can be a hole. The hole is preferably not substantially larger than the test opening to provide a good seal between the fabric and the test opening. Another example of a window is a substantially transparent portion of the top plate on top of the test opening. Another example of a window is a semi-transparent portion of the top plate on top of the test opening.

In some embodiments, at least a portion of the window in the top plate is configured to be aligned with the test opening via an alignment structure. The alignment structure helps align the window in the top plate substantially above the test opening. This allows observation of the fabric atop the test opening during testing. In some embodiments, the alignment structure is a guide-pin with an alignment hole. An exemplary embodiment is shown in FIG. 4. Window 116 in top plate 112 is aligned with test opening 114 via two guide pins (118a and 118b) and alignment holes (112a and 112b). In some embodiments, the alignment structure includes a stop-block, magnet, c-clamp, an elastic band, and/or one or more nuts/bolts.

In some embodiments, the top plate and the bottom supporting structures are aligned by magnets. In one embodiment, two pairs of magnets are embedded and fixed on each side of the top plate and bottom supporting structures, as illustrated in FIG. 5. The magnets (e.g., 502a/502b and 504a/504b) are arranged so that they are attracted to each other and will bring the two structures closer to each other. Once the two magnets are in contact, During this process, the two structures are compressed against each other and aligned automatically. In some embodiments, the minimum force the magnets applied is greater than or equal to about 5N.

In some embodiments, the supporting structure and top plate are compressed via a compression mechanism. The compression between the supporting structure and the top plate provides seal between the test opening, top plate and the fabric, and facilitates the accuracy of the hydrostatic pressure measurements. In one aspect, the weight of the top plate can act as a compression mechanism when the top plate is placed atop the supporting structure or/and the fabric. In another aspect, the compression mechanism can be a c-clamp, a nut and a bolt, an elastic band, or a magnet. In some embodiments, at least two supporting ends are providing compression and support for the top plate. An exemplary embodiment is shown in FIG. 2B. Two supporting ends, 102a and 102b, are supporting the top plate and providing compression between the test opening, fabric, and the top plate.

In some embodiments, the test opening further comprises a sealing structure configured to form a seal between the fabric and the test opening. The seal between the fabric and the test opening facilitates the accuracy of hydrostatic pressure measurements. The sealing structure can take many forms. In some embodiments, the sealing structure is a gasket. In some embodiments, the sealing structure comprises a compressible material. The compressible material helps to form the seal between the fabric and the test opening. In some embodiments, the compressible material is silicone, rubber, or foam. In another aspect, the sealing is formed to help the seal of the test sample between the top plate and the test opening. The pressure between the test opening and the top plate provides enough force to create a liquid-tight seal for the test sample. In some embodiments, the sealing can be formed via the weight of the top plate when it is placed on top of the fabric and/or the test opening. In some embodiments, the alignment structures can function as a sealing mechanism to compress the top plate, the fabric and the test opening, in addition to aligning the window in the top plate with at least a portion of the test opening. In some embodiments, the compression mechanism can function as a sealing mechanism to compress the top plate, the fabric and the test opening and to form a liquid-tight seal for the test sample.

The test opening can be of different sizes. The different sizes of the test opening are used for testing different samples and various areas of a sample. In some embodiments, the test opening has a diameter of between about 1 mm and about 10 cm. The device in the present disclosure is capable of testing small sizes of samples without cutting the sample before testing. In some embodiments, the test opening has a diameter between about 1 mm and about 2 mm, between about 2 mm and about 3 mm, between about 3 mm and about 10 mm, between about 10 mm and about 100 mm, between about 10 mm and about 10 cm, between about 5 mm and about 10 mm, between about 1 cm and about 2 cm, between about 1 cm and about 3 cm, between about 1 cm and about 4 cm, between about 1 cm and about 5 cm, between about 1 cm and about 6 cm, between about 1 cm and about 7 cm, between about 1 cm and about 8 cm, between about 1 cm and about 9 cm, between about 1 cm and about 10 cm, or between about 5 cm and about 10 cm. It is contemplated that, in embodiments in which the test opening is not circular, the exemplary test opening diameter sizes described above may instead recite a length and/or width of the test opening.

The reservoir can take any shape that allows it to hold liquid. In some embodiments, the reservoir is a cylinder. In one aspect, the reservoir further comprises an indication of the liquid level inside the reservoir. In some embodiments, a ruler can be added to the reservoir to determine the liquid level and/or liquid volume inside the reservoir. In some embodiments, the reservoir has pre-calibrated markings to determine the liquid level and/or liquid volume inside the reservoir. In some embodiments, the reservoir has at least a portion that is substantially transparent, or semi-transparent, and the portion allows observation of the liquid level and/or liquid volume inside the reservoir.

Any liquid that remains liquid form under testing conditions can be used in the device in the present disclosure. Typically, water is used to test the hydrostatic pressure resistance of a sample. Other examples of liquids include without limitation alcohol, hexane, saline solution, oil, or a biological fluid (e.g., blood, sweat, or urine).

Advantageously, the devices of the present disclosure can test the hydrostatic pressure resistance of a sample without the need of a power source. The device herein utilizes gravity to drive the liquid flow upon the elevation of the reservoir. For example, the device herein can be used without a pump or a motor to apply the hydrostatic pressure. However, in some embodiments, a motor or other power source can optionally be used to elevate the reservoir.

In some embodiments, a device of the present disclosure can apply a hydrostatic pressure of up to about 5 kpa. For example, in some embodiments, a device of the present disclosure applies a hydrostatic pressure of about 0.5 kpa or more, about 1 kpa or more, about 1.5 kpa or more, about 2 kpa or more, about 2.5 kpa or more, about 3 kpa or more, about 3.5 kpa or more, about 4 kpa or more, about 4.5 kpa or more, and/or up to about 5 kpa.

A variety of materials may suitably be used for components of the present device. In some embodiments, the inlet tube comprises polyurethane, vinyl, or plastic. In some embodiments, the top plate comprises wood, acrylic, polycarbonate, metal, or glass. In some embodiments, the supporting structure comprises wood, acrylic, polycarbonate, metal, or glass. In some embodiments, the test opening comprises wood, acrylic, polycarbonate, metal, or glass. In some embodiments, the reservoir comprises glass, PVC, metal, PET, or polycarbonate. In some embodiments, the connection tube comprises plastic or metal. In some embodiments, the metal is aluminum, an aluminum alloy, copper, brass, or steel (e.g., stainless steel). In some embodiments, an edge of the test opening comprises wood, acrylic, polycarbonate, metal, or glass.

Components of the device can be of many shapes. For example, in some embodiments, the test opening is circular, square, triangular, or hexagonal. The connection tube can be cylindrical, a rectangular or triangular prism, or a dumbbell shapes. It is to be understood that these shapes are exemplary and not intended to be limiting, as other shapes can readily be utilized by one of ordinary skill in the art.

### II. Methods of Using Portable Hydrostatic Testers

Certain aspects of the present disclosure relate to methods of testing the hydrostatic pressure resistance of a fabric with the portable hydrostatic testers of the present disclosure. Exemplary methods of testing the hydrostatic pressure resistance are set forth below, but the skilled artisan will appreciate that various liquids, components of various materials, and various indications of hydrostatic pressure via the liquid column and/or reservoir may be used to test the hydrostatic pressure resistance of a fabric, depending upon the specific property of the fabric, without departing form the scope of the present disclosure.

In some embodiments, a method for testing the hydrostatic pressure resistance of a fabric, comprising a) providing a device, comprising: (i) a supporting structure comprising a test opening, wherein the supporting structure is configured to support the fabric; (ii) a connection tube comprising a first end and a second end, wherein the first end is connected to the test opening; (iii) a liquid column connected to the second end of the connection tube; and (iv) a reservoir containing a liquid; b) placing the fabric on the test opening; and c) elevating the reservoir such that the liquid fills the connection tube and at least a portion of the liquid column, wherein the liquid is forced by gravity through the test opening via the connection tube at a hydrostatic pressure dependent upon the volume of liquid in the connection tube and the liquid column. Upon elevation of the reservoir, the liquid flows through the connection tube and the liquid column, and the liquid applies a hydrostatic pressure on the fabric through the test opening as the liquid level and/or volume increases within the liquid column. In some embodiments, the liquid is driven by the gravity to flow from the reservoir to the connection tube via an inlet tube. In some embodiments, the inlet tube further comprises a valve configured to adjust the flow of the liquid from the reservoir. For example, the valve may be opened after or concurrently with elevation of the reservoir to permit liquid flow through the inlet tube. To better utilize the capacity of the reservoir, in some embodiments, the inlet tube is connected to a portion of the reservoir that is lower than its geometric center. In some embodiments, the liquid is water.

In some embodiments, the method further comprises recording the hydrostatic pressure within one or both of the liquid column and the reservoir after step c). The recording can be made upon indications of hydrostatic pressure within one or both of the liquid column and the reservoir. The indications can be of many forms. For example, the liquid column and/or the reservoir have a pre-calibrated marking to indicate the liquid level and/or liquid volume within the liquid column and/or the reservoir (e.g., the hydrostatic pressure). Alternatively, the liquid column and/or the reservoir have at least a portion that is substantially transparent or semi-transparent. That portion allows the liquid level and/or the liquid volume to be observed within the liquid column and/or the reservoir, thereby indicating the hydrostatic pressure. In some embodiments, a ruler can be used to indicate the hydrostatic pressure.

In some embodiments, the method further comprises forming a seal between the fabric and the test opening between steps b) and c). This can be achieved by using a sealing mechanism as discussed above. In some embodiments, the sealing mechanism is a gasket. In some embodiments, the sealing structure comprises a compressible material. The compressible material can be silicone, rubber, or foam. In some embodiments, a compression mechanism can form a seal between the fabric and the test opening. The compression mechanism can be a c-clamp, a nut and a bolt, elastic band, or a magnet. In some embodiments, the weight of the top plate can form a seal between the fabric and the test opening.

Positions of the device and its components impact the accuracy of the hydrostatic pressure resistance measurements. In some embodiments, the hydrostatic tester is positioned on a substantially horizontal surface. In some embodiments, the liquid column is in a substantially vertical position.

In some embodiments, the method further comprising adding a top plate between steps b) and c), wherein the top plate is configured to be positioned atop one or both of the fabric and the supporting structure. In some embodiments, the top plate comprises a window that allows observation of the fabric atop the test opening during testing. The window in the top plate can be a hole or a portion of the top plate that is substantially transparent or semi-transparent. The window is aligned with at least a portion of the test opening. The alignment of the window and the test opening can be achieved by an alignment structure. The alignment structure can be a guide pin, a stop-block or magnet.

The methods described herein allow testing of the hydrostatic pressure resistance of a sample without the need for a power source. Upon the elevation of the reservoir, gravity drives the liquid to flow from the reservoir to the test opening. Therefore, the methods herein do not require a power source, such as a pump or a motor.

The methods described herein also allow testing of a full-sized product without cutting the sample before testing or damaging the sample. In addition, the methods herein also enable testing of samples of small sizes. For example, the test opening can have a diameter of between about 1 mm and about 10 cm. This also enables testing of a small area in a bigger sample. Furthermore, after completion of testing one area, the sample can be moved to allow testing of a different area on the same sample. For example, fabric 300 shown in FIGS. 3A & 3B can be shifted after testing to test another area of the fabric.

### EXAMPLES

The present disclosure will be more fully understood by reference to the following example. It should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example: Portable Hydrostatic Testing

A portable hydrostatic tester of the present disclosure is used to determine hydrostatic pressure resistance of multiple areas of a fabric. For hydrostatic pressure testing, the reservoir of the tester (e.g., reservoir 108) is filled with enough deionized (DI) water for conducting the measurement. The portable hydrostatic tester is placed on a flat surface and calibrated before testing: the reservoir is adjusted to ensure that the water level meets the edge of the testing opening (without overflowing) when the pressure reading from the liquid column is at “0” pa. The top plate (e.g., 112) is loosened from the supporting structures (e.g., 102), and the testing sample (e.g., fabric 300) is slipped through the gap between the two structures. The surface of the fabric to be treated should be facing down towards the test opening. The position of the fabric sample is adjusted until the opening on the top plate (e.g., viewing window 116) is aligned with the area to be tested. Then the top plate and the supporting structures are tightened together to provide sufficient seal around the testing opening and testing sample. The reservoir is then raised to a certain height to provide enough hydrostatic pressure and close attention is paid to the observation window at the top plate. The rising rate of the liquid level in the liquid column can be adjusted by the valve in the inlet tube. Once leakage is observed through the fabric (e.g., via a viewing window in the top plate), the pressure reading at the liquid column is quickly recorded which corresponds to the maximum hydrostatic pressure of this area of the sample. See, e.g., FIG. 3B. Multiple areas on the fabric sample can be measured and recorded to provide a mapping of the hydrostatic pressures without damaging the sample.

Although the foregoing descriptions and examples have been described in some detail by way of illustration and example for purposes of clarity of understanding, the descriptions and examples should not be construed as limiting the scope of the present disclosure.

Exemplary devices and methods are set out in the following non-limiting items:

Item 1. A device for hydrostatic testing of a fabric, comprising:

(1) a supporting structure comprising a test opening, wherein the supporting structure is configured to support the fabric;
(2) a connection tube comprising a first end and a second end, wherein the first end is connected to the test opening;
(3) a liquid column connected to the second end of the connection tube; and
(4) a reservoir configured to contain a liquid, wherein the reservoir is connected to one or both of the liquid column and the connection tube, and wherein the reservoir is configured such that the liquid is forced by gravity through the test opening via the connection tube at a hydrostatic pressure dependent upon the volume of water contained in the connection tube and liquid column.

Item 2. The device of item 1, wherein the reservoir is connected to one or both of the liquid column and the connection tube via an inlet tube.

Item 3. The device of item 2, wherein a junction between the inlet tube and the liquid column and/or the connection tube is positioned lower than the test opening.

Item 4. The device of item 2 or item 3, wherein the inlet tube further comprises a valve configured to adjust the flow of the liquid from the reservoir.

Item 5. The device of any one of items 2 to 4, wherein the inlet tube comprises polyurethane, vinyl, polypropylene, rubber, soft polymer, or plastic.

Item 6. The device of any one of items 1 to 5, wherein the liquid column is configured such that the level of the liquid within the liquid column indicates the hydrostatic pressure.

Item 7. The device of any one of items 1 to 6, wherein at least a portion of the liquid column is substantially transparent.

Item 8. The device of any one of items 1 to 7, wherein the liquid column further comprises an indication of hydrostatic pressure.

Item 9. The device of any one of items 1 to 8, wherein the liquid column is a cylinder.

Item 10. The device of any one of items 1 to 8, wherein the liquid column is a rectangular prism.

Item 11. The device of any one of items 1 to 10, wherein the liquid column has a first end and a second end, wherein the first end is open, and wherein the second end is connected to the second end of the connection tube.

Item 12. The device of any one of items 1 to 11 further comprising a top plate, wherein the top plate is configured to be positioned atop one or both of the fabric and the supporting structure.

Item 13. The device of item 12, wherein the top plate comprises a window for viewing the fabric.

Item 14. The device of item 13, wherein at least a portion of the window is configured to be aligned with the test opening via an alignment structure.

Item 15. The device of item 14, wherein the alignment structure comprises a guide-pin with an alignment hole, a stop-block, or a magnet.

Item 16. The device of any one of the items 12 to 15, wherein the supporting structure and top plate are compressed via a compression mechanism.

Item 17. The device of item 16, wherein the compression mechanism comprises a c-clamp, a nut and a bolt, an elastic band, or a magnet.

Item 18. The device of any one of items 12 to 17, wherein the top plate comprises wood, acrylic, polycarbonate, metal, or glass.

Item 19. The device of any one of items 1 to 18, wherein the supporting structure comprises wood, acrylic, polycarbonate, metal, or glass.

Item 20. The device of any one of items 1 to 19, wherein the test opening further comprises a sealing structure configured to form a seal between the fabric and the test opening.

Item 21. The device of item 20, wherein the sealing structure comprises a gasket.

Item 22. The device of any one of items 20 or 21, wherein the sealing structure comprises a compressible material.

Item 23. The device of item 22, wherein the compressible material comprises silicone, rubber, or foam.

Item 24. The device of any one of items 1 to 23, wherein the test opening has a diameter of between about 1 mm and about 10 cm.

Item 25. The device of item 24, wherein the test opening has a diameter between about 2 mm and about 3 mm.

Item 26. The device of any one of items 1 to 25, wherein an edge of the test opening comprises wood, acrylic, polycarbonate, metal, or glass.

Item 27. The device of any one of items 1 to 26, wherein the reservoir is a cylinder.

Item 28. The device of any one of items 1 to 27, wherein the reservoir further comprises an indication of the liquid level inside the reservoir.

Item 29. The device of any one of items 1 to 28, wherein the reservoir comprises glass, PVC, metal, PET, or polycarbonate.

Item 30. The device of any one of items 1 to 29, wherein the connection tube comprises plastic or metal.

Item 31. The device of item 30, wherein the metal is aluminum, aluminum alloy, copper, brass, or steel.

Item 32. The device of any one of items 1 to 31, wherein the liquid is water.

Item 33. The device of any one of items 1 to 32, wherein the device lacks a pump or motor.

Item 34. The device of any one of items 1 to 33, wherein the liquid column contains a liquid.

Item 35. A method for testing the hydrostatic pressure resistance of a fabric, comprising:

a) providing a device, the device comprising:
(i) a supporting structure comprising a test opening, wherein the supporting structure is configured to support the fabric;
(ii) a connection tube comprising a first end and a second end, wherein the first end is connected to the test opening;
(iii) a liquid column connected to the second end of the connection tube; and
(iv) a reservoir containing a liquid;
   b) placing the fabric on the test opening; and
   c) elevating the reservoir such that the liquid fills the connection tube and at least a portion of the liquid column, wherein the liquid is forced by gravity through the test opening via the connection tube at a hydrostatic pressure dependent upon the volume of liquid in the connection tube and the liquid column.

Item 36. The method of item 35, further comprising recording the liquid level within one or both of the liquid column and the reservoir after step c.

Item 37. The method of item 35 or item 36, further comprising forming a seal between the fabric and the test opening between steps b and c.

Item 38. The method of item 37, wherein the seal is formed via a sealing structure.

Item 39. The method of item 38, wherein the sealing structure comprises a gasket.

Item 40. The method of item 38 or item 39, wherein the sealing structure comprises a compressible material.

Item 41. The method of item 40, wherein the compressible material comprises silicone, rubber, or foam.

Item 42. The method of any one of items 35 to 41, wherein the device is positioned on a substantially horizontal surface.

Item 43. The method of any one of items 35 to 42, wherein the liquid column is in a substantially vertical position.

Item 44. The method of any one of items 35 to 43, wherein the liquid is water.

Item 45. The method of any one of items 35 to 44, wherein the reservoir is connected to one or both of the liquid column and the connection tube via an inlet tube.

Item 46. The method of item 45, wherein a junction between the inlet tube and the liquid column and/or the connection tube is positioned lower than the test opening.

Item 47. The method of any one of items 45 or 46, wherein the inlet tube further comprises a valve configured to adjust the flow of the liquid from the reservoir.

Item 48. The method of any one of items 45 to 47, wherein the inlet tube comprises polyurethane, vinyl, polypropylene, rubber, soft polymer, or plastic.

Item 49. The method of any one of items 35 to 48, wherein the liquid column is configured such that the level of the liquid within the liquid column indicates the hydrostatic pressure.

Item 50. The method of any one of items 35 to 49, wherein at least a portion of the liquid column is substantially transparent.

Item 51. The method of any one of items 35 to 50, wherein the liquid column further comprises an indication of hydrostatic pressure.

Item 52. The method of any one of items 35 to 51, wherein the liquid column is a cylinder.

Item 53. The method of any one of items 35 to 51, wherein the liquid column is a rectangular prism.

Item 54. The method of any one of items 35 to 53, wherein the liquid column has a first end and a second end, wherein the first end is open, and wherein the second end is connected to the second end of the connection tube.

Item 55. The method of any one of items 35 to 54, further comprising a top plate, wherein the top plate is configured to be positioned atop one or both of the fabric and the supporting structure.

Item 56. The method of item 55, wherein the top plate comprises a window for viewing the fabric.

Item 57. The method of item 56, wherein at least a portion of the window is configured to be aligned with the test opening via an alignment structure.

Item 58. The method of item 57, wherein the alignment structure comprises a guide-pin with an alignment hole, a stop-block, or a magnet.

Item 59. The method of any one of items 55 to 58, wherein the supporting structure and top plate are compressed via a compression mechanism.

Item 60. The method of item 59, wherein the compression mechanism comprises a c-clamp, a nut and a bolt, an elastic band, or a magnet.

Item 61. The method of any one of items 55 to 60, wherein the top plate comprises wood, acrylic, polycarbonate, metal, or glass.

Item 62. The method of any one of items 35 to 61, wherein the supporting structure comprises wood, acrylic, polycarbonate, metal, or glass.

Item 63. The method of any one of items 35 to 62, wherein the test opening has a diameter of between about 1 mm and about 10 cm.

Item 64. The method of item 63, wherein the test opening has a diameter between about 2 mm and about 3 mm.

Item 65. The method of any one of items 35 to 64, wherein an edge of the test opening comprises wood, acrylic, polycarbonate, metal, or glass.

Item 66. The method of any one of items 35 to 65, wherein the reservoir is a cylinder.

Item 67. The method of any one of items 35 to 66, wherein the reservoir further comprises an indication of the liquid level inside the reservoir.

Item 68. The method of any one of items 35 to 67, wherein the reservoir comprises glass, PVC, metal, PET, or polycarbonate.

Item 69. The method of any one of items 35 to 68, wherein the connection tube comprises plastic or metal.

Item 70. The method of item 69, wherein the metal is aluminum, aluminum alloy, copper, brass, or steel.

Item 71. The method of any one of items 35 to 70, wherein the device lacks a pump or motor.

Item 72. The method of any one of items 35 to 71, wherein the liquid column contains a liquid.

What is claimed is:

1. A device for hydrostatic testing of a fabric, comprising:
(1) a supporting structure comprising a test opening, wherein the supporting structure is configured to support the fabric;
(2) a connection tube comprising a first end and a second end, wherein the first end is connected to the test opening;
(3) a liquid column connected to the second end of the connection tube; and
(4) a reservoir configured to contain a liquid, wherein the reservoir is connected to one or both of the liquid column and the connection tube, and wherein the reservoir is configured such that the liquid is forced by gravity through the test opening via the connection tube at a hydrostatic pressure dependent upon the volume of water contained in the connection tube and liquid column.

2. The device of claim 1, wherein the reservoir is connected to one or both of the liquid column and the connection tube via an inlet tube.

3. The device of claim 1, wherein the liquid column is configured such that the level of the liquid within the liquid column indicates the hydrostatic pressure.

4. The device of claim 1, wherein the liquid column further comprises an indication of hydrostatic pressure.

5. The device of claim 1, further comprising a top plate, wherein the top plate is configured to be positioned atop one or both of the fabric and the supporting structure.

6. The device of claim 5, wherein the top plate comprises a window for viewing the fabric.

7. The device of claim 6, wherein at least a portion of the window is configured to be aligned with the test opening via an alignment structure.

8. The device of claim 5, wherein the supporting structure and top plate are compressed via a compression mechanism.

9. The device of claim 1, wherein the test opening further comprises a sealing structure configured to form a seal between the fabric and the test opening.

10. The device of claim 1, wherein the device lacks a pump or motor.

11. A method for testing the hydrostatic pressure resistance of a fabric, comprising:
a) providing a device, the device comprising:
(i) a supporting structure comprising a test opening, wherein the supporting structure is configured to support the fabric;
(ii) a connection tube comprising a first end and a second end, wherein the first end is connected to the test opening;
(iii) a liquid column connected to the second end of the connection tube; and
(iv) a reservoir containing a liquid;
b) placing the fabric on the test opening; and
c) elevating the reservoir such that the liquid fills the connection tube and at least a portion of the liquid column, wherein the liquid is forced by gravity through the test opening via the connection tube at a hydrostatic pressure dependent upon the volume of liquid in the connection tube and the liquid column.

12. The method of claim 11, further comprising forming a seal between the fabric and the test opening between steps b and c.

13. The method of claim 11, wherein the reservoir is connected to one or both of the liquid column and the connection tube via an inlet tube.

14. The method of claim 11, wherein the liquid column is configured such that the level of the liquid within the liquid column indicates the hydrostatic pressure.

15. The method of claim 11, wherein the liquid column further comprises an indication of hydrostatic pressure.

16. The method of claim 11, further comprising a top plate, wherein the top plate is configured to be positioned atop one or both of the fabric and the supporting structure.

17. The method of claim 16, wherein the top plate comprises a window for viewing the fabric.

18. The method of claim 17, wherein at least a portion of the window is configured to be aligned with the test opening via an alignment structure.

19. The method of claim 16, wherein the supporting structure and top plate are compressed via a compression mechanism.

20. The method of claim 11, wherein the device lacks a pump or motor.
